(19) United States
(12) Patent Application Publication
(10) Pub. No.: US 2010/0152487 A1
(43) Pub. Date: Jun. 17, 2010

(54) AGENT AND METHOD FOR STABILIZING MEMBRANE PROTEIN

(75) Inventors: Yuichi Sugiyama, Tokyo (JP); Hisamitsu Hayashi, Tokyo (JP); Takuya Kato, Tokyo (JP)

Correspondence Address: PEARNE & GORDON LLP, 1801 EAST 9TH STREET, SUITE 1200, CLEVELAND, OH, 44114-3108, US

(73) Assignees: Yuichi SUGIYAMA, Tokyo (JP); Hisamitsu HAYASHI, Tokyo (JP)

(21) Appl. No.: 12/667,161

(22) PCT Filed: May 19, 2008
(86) PCT No.: PCT/JP2008/001239

§ 371 Date: Jan. 25, 2010

(30) Foreign Application Priority Data
Jul. 12, 2007 (JP) 2007-182846

(51) Int. Cl. C07C 57/30; C07C 53/126

(52) U.S. Cl. 562/496; 562/512

(57) ABSTRACT

[Problems to be Solved] It is an object of the present invention to provide an agent and a method for maintaining and promoting the stability of a membrane protein, and in particular, a transporter (for example, an ABC transporter) on a cell membrane.

[Solution] The present invention provides: a method of treating a cell with carboxylic acid including 4-phenylbutyric acid or a derivative thereof to suppress the decomposition of a protein existing on a cell membrane, so as to maintain and promote the stability of the protein on the membrane; and an agent for stabilizing a membrane protein, which comprises carboxylic acid including 4-phenylbutyric acid or a derivative thereof.

### TECHNICAL FIELD

The present invention relates to an agent and a method for stabilizing a membrane protein. More specifically, the present invention relates to an agent and a method for stabilizing a transporter.

### BACKGROUND ART

Membrane protein exists in a biological membrane. It functions as a receptor, a transporter (transporting body), or a membrane enzyme, and it also acts to maintain and promote the structure or movement of a membrane.

Among such membrane proteins, for example, a transporter has been known to play an important role in the maintenance of an intracellular environment, and its functional mutation causes various diseases. In a broad sense, a transporter is a protein, which exists in a biological membrane and mediates material transport across the biological membrane. In particular, a transporter, which is associated with the uptake and efflux of drugs, conducts transport of organic anion or organic cation, and thus, it plays an essential role for a living body, such as the absorption of drugs and elimination. This transporter exists in cells that form various types of organs such as the liver, intestinal canal, kidney, and brain.

If the function of such transporter associated with the uptake of drugs and elimination is weakened, a toxic substance may be accumulated in cells and it may cause the onset of a serious disease in some cases. For example, progressive familial intrahepatic cholestasis 2 (PFIC2) is a hereditary disease caused by genetic deficiency in the expression of a bile salt export pump (BSEP/ABCB11) existing in the canalicular membrane. If surgical procedures such as liver transplantation are not performed on this intractable disease, hepatocirrhosis would be developed, and life would be threatened.

BSEP is classified into an ATP-binding cassette transmembrane transporter (an ABC transporter) located on the canalicular membrane, and it acts to eliminate bile acid (taurocholic acid). If the functions of BSEP are impaired for any reason, bile acid would be accumulated in liver cells, and as a result, the liver cells would be greatly damaged by the toxicity of a high concentration of bile acid. From the results of the genetic analysis of PFIC2 patients, it has become clear that there are various BSEP gene mutations, such as missense mutations, immature termination, frameshift, and splicing junction mutations. Among others, two missense mutations in a second intracellular loop and a first ATP-binding domain, E297G and D482G, are often found in PFIC2 patients (Non-Patent Document 1).

In the case of transporters other than BSEP as well, a reduction in activity due to abnormality in the functions thereof often leads to the onset of disease. Thus, it is considered that the effective maintenance of the activity of such transporter would contribute to the development of an effective method for treating relevant diseases. To date, it has been reported that a mutant (CFTRΔF508) of a cystic fibrosis transmembrane conductance regulator (CFTR/ABCC7; chlorine ion channel), which is characterized in that its expression on a cell membrane is significantly decreased, recovers its expression on the cell membrane by the action of 4-phenylbutyric acid (4PBA) (Non-Patent Document 2). However, a mechanism for maintaining the stability of a transporter on a cell membrane, which includes the recovery of a transporter on a cell membrane, has not yet been clarified.

### Non-Patent Document 1: Hayashi et al., Hepatology, 41: 916-924, 2005

Non-Patent Document 2: Ronald et al., Am J. Respir. Crit. Care. Med., 157: 484-490, 1998

### DISCLOSURE OF THE INVENTION

### Problems to be Solved by the Invention

It is an object of the present invention to provide an agent and a method for stabilizing a membrane protein, and in particular, a transporter (for example, an ABC transporter) on a cell membrane.

### Means for Solving the Problems

As a result of intensive studies directed towards maintaining and promoting the functions of a transporter, the present inventors have found that carboxylic acid including 4-phenylbutyric acid (which is also referred to as “4PBA”) suppresses the decomposition of BSEP existing on a canalicular membrane, thereby completing the present invention. Specifically, the present invention relates to an agent for stabilizing a membrane protein, which is characterized in that it comprises, as an active ingredient, a compound represented by the following Formula (1), a pharmaceutically acceptable salt thereof, or a hydrate thereof:

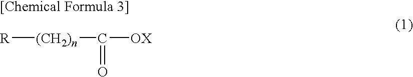

(wherein R represents a hydrogen atom, a methyl group, or a phenyl group non-substituted or having a substituent; X represents a hydrogen atom or an alkali metal atom; and n represents an integer of 1 to 8).

Alternatively, the present invention relates to an agent for stabilizing a membrane protein, which is characterized in that it comprises, as an active ingredient, a compound represented by the following Formula (2), a pharmaceutically acceptable salt thereof, or a hydrate thereof:

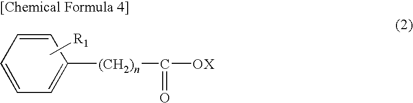

(wherein R₁ represents a hydrogen atom or an alkyl group containing 1 to 5 carbon atoms; X represents a hydrogen atom or an alkali metal atom; and n represents an integer of 1 to 8).

Moreover, the present invention relates to a method for stabilizing a membrane protein using the above-described stabilizing agent.

Furthermore, the present invention relates to a cholagogue comprising the above-described stabilizing agent.

### EFFECTS OF THE INVENTION

Using the stabilizing agent according to the present invention, the stability of a membrane protein, and in particular, a transporter (for example, an ABC transporter) on a membrane can be maintained or promoted.

In addition, using a drug containing the stabilizing agent of the present invention, there can be provided an agent and a method for treating diseases (for example, cholestatic disease) developed as a result of abnormality in the functions of a transporter (for example, an ABC transporter) (for example, BSEP).

### BRIEF DESCRIPTION OF THE DRAWINGS

FIG. 1 shows the time- and concentration-dependent effects of a 4PBA treatment on MDCKII cells that had expressed BSEP. FIG. 1A shows the time-dependent effects of APBA on the expression of BSEP. The MDCKII cells that had expressed wild-type, E297G and D482G BSEPs were each treated with 1 mM 4PBA at time points shown in the figure, so that a crude membrane fraction was prepared. The obtained sample (40 μg) was subjected to 6% SDS-PAGE, and Western blotting was then carried out. FIG. 1B shows the concentration-dependent effects of APBA on the expression of BSER MDCKII cells that had expressed wild-type, E297G and D482G BSEPs were each treated with 4PBA having the concentrations shown in the figure for 24 hours, so that a crude membrane fraction was prepared. The obtained sample (40 μg) was subjected to 6% SDS-PAGE, and Western blotting was then carried out.

FIG. 2 shows the effects of a 4PBA treatment on the expression of BSEP on the surfaces of MDCKII cells. Before carrying out the experiment, the MDCKII cells that had each expressed wild-type, E297G and D482G BSEPs, and GFP were treated for 24 hours in the presence or absence of 1 mM 4PBA. The expression of BSEP on the cell surface was measured by a transcellular transport assay and a cell surface biotinylation method. FIG. 2A shows a change over time of the transcellular transport of [³H] TC that got across a monolayer culture of MDCKII. The filled circle and the filled square represent MDCKII cells treated in the presence of 4PBA, and the open circle and the open square represent MDCKII cells treated in the absence of 4PBA. The circle represents a transcellular transport in the direction from the base side to the apical side, and the square represents a transcellular transport in the direction from the apical side to the base side. Each point and vertical bar indicate the mean value of 3 times of measurement values±a standard error. FIG. 2B shows the state of the transcellular transport of [³H] TC that got across the apical membrane of a monolayer culture of MDCKII. There was measured the clearance of the transcellular transport of [³H] TC that got across each of the apical membranes of monolayer cultures of MDCKII cells that had expressed wild-type, E297G and D482G BSEPs, and GFP. Each bar indicates the mean value of 3 times of measurement values±a standard error. A significant difference from a control by a Student's test (*P<0.05; **P<0.01)

FIG. 3 shows the results obtained by measuring cell surface expression according to biotinylation analysis. A cell surface fraction was isolated by a biotinylation method. The obtained sample was subjected to 6% SDS-PAGE, and Western blotting was then carried out.

FIG. 4 shows the results obtained by analyzing the influence of 4PBA on transcription and translation with respect to BSEP expression in MDCKII cells. FIG. 4A shows the results obtained by measuring the expression level of BSEP mRNA according to real-time PCR. The expression level of BSEP in each reaction was standardized by the expression level of GAPDH. Each bar indicates the mean value of 3 times of measurement values±a standard error. FIGS. 4B and 4C show the results obtained by analyzing the upregulation of BSEP by 4PBA under conditions of (B) transcription inhibition by actinomycin D and (C) translation inhibition by cycloheximide. MDCKII cells that had expressed wild-type, E297G and D482G BSEPs were treated in the presence or absence of 1 mM 4PBA, under conditions in the presence or absence of actinomycin D (5 μg/ml), or in the presence or absence of cycloheximide (20 μg/ml), for 6 hours (in the case of E297G and D482G BSEPs), or for 8 hours (a wild-type BSEP). 40 μg of a sample was subjected to 6% SDS-PAGE, and Western blotting was then carried out.

FIG. 5 shows the results obtained by examining the influence of a 4PBA treatment on the process of maturation of BSEP in MDCKII cells. FIG. 5A shows the results obtained by examining the course of maturation of BSEP. MDCKII cells that had expressed wild-type, E297G, and D482G BSEPs were treated in the presence or absence of 5 μg/ml BFA and with or without the addition of 1 mM 4PBA. In order to confirm a state in which an immature low-molecular-weight BSEP accumulated due to BFA was converted to a mature high-molecular-weight BSEP, BFA was washed out over time in the presence or absence of 4PBA, so that a crude membrane fraction was prepared. 40 μg out of the obtained sample was subjected to 6% SDS-PAGE, and Western blotting was then carried out. FIG. 5B shows the results obtained by quantifying the band density of the mature BSEP shown in FIG. 5A. Such band density was quantified by Image Gauge software. The filled circle represents a signal strength obtained from the band of the mature BSEP in the presence of 4PBA, and the open circle represents a signal strength obtained from the band of the mature BSEP in the absence of 4PBA.

FIG. 6 shows the results obtained by examining the effects of 4PBA on the decomposition rate of BSEP existing on the surfaces of MDCKII cells. FIG. 6A shows the results obtained by analyzing the decomposition rates of wild-type and E297G BSEPs existing on the surfaces of the cells. The MDCKII cells that had expressed wild-type and E297G BSEPs were treated in the presence or absence of 1 mM 4PBA. The cell surface was biotinylated, and the cells were then cultured in the presence or absence of 1 mM 4PBA at 37° C. for the period of time shown in the figure. The remaining biotinylated protein was isolated using streptavidin beads, and was then separated by 6% SDS-PAGE, followed by Western blotting. FIG. 6B shows the results obtained by quantifying the band densities of the wild-type and E297G BSEPs shown in FIG. 6A. The band density was quantified by Image Gauge software, and it was indicated with a percentage with respect to the strength at 0 (zero) hour. The filled circle represents the value of each BSEP remaining on the surfaces of MDCKII cells treated with 4PBA, and the open circle represents the value of each BSEP remaining on the surfaces of MDCKII cells that were not treated with 4PBA. Each bar indicates the mean value of 3 times of measurement values±a standard error. By the same method, P-gp and DPPIV were also measured in terms of the band density.

FIG. 7 shows the results obtained by examining the effects of 4PBA on the decomposition rate of a D482G BSEP existing on the surfaces of MDCKII cells. FIG. 7A shows the results obtained by analyzing the expression of the D482G BSEP existing on the surfaces of the cells under low temperature conditions (27° C.). The MDCKII cells that had expressed the D482G BSEP were cultured at 27° C. for 24 hours, and the cell surface was then biotinylated. The biotinylated protein was isolated using streptavidin beads, and it was then separated by 6% SDS-PAGE, followed by Western blotting. FIG. 7B shows the results obtained by examining the decomposition rate of the D482G BSEP existing on the surfaces of the cells. The MDCKII cells that had expressed the D482G BSEP were cultured in the presence or absence of 1 mM 4PBA at 27° C. for 24 hours. The sample prepared by the same method as that in FIG. 7A was separated by SDS-PAGE, and Western blotting was then carried out. FIG. 7C shows the results obtained by quantifying the band density of the D482G BSEP shown in FIG. 7D. The band density was quantified by the same method as that in FIG. 7B, and the results were then shown in the form of a graph.

FIG. 8 shows the results obtained by analyzing the influence of 4PBA on the expression of BSEP on the canalicular membrane of an SD rat. Before carrying out the experiment, 4PBA or a vehicle was administered to a male SD rat (6- to 7-week-old) via a tube at a dosage of 0.6 g/kg/day for 10 days. Such 4PBA or vehicle was administered in 3 divided doses. FIG. 8A shows the time-dependent uptake of [³H] TC by CMVs. That is, CMVs prepared from each of the SD rats treated with 4PBA (filled circle) and a vehicle (open circle) was incubated at 37° C. in the presence of 5 mmol/L ATP or AMP. The concentration of [³H] TC was 1 μM. The uptake of [³H] TC was calculated by subtracting the value obtained in the presence of AMP from the value in the presence of ATP. Each point and horizontal bar indicate the mean value of 3 times of experiments±a standard error. FIG. 8B shows the Hofstee plot of the uptake of [³H] TC by CMVs. That is, CMVs prepared from each of the SD rats treated with 4PBA (filled circle) and a vehicle (open circle) was incubated for 0.5 minutes or 1 minute at 37° C. in the presence of 5 mmol/L ATP or AMP. The concentration of [³H] TC was 1 μM. The uptake of [³H] TC was calculated by subtracting the value obtained in the presence of AMP from the value in the presence of ATR Each point and horizontal bar indicate the mean value of 3 times of experiments±a standard error. FIG. 8C shows the results of Western blotting performed on CMVs. That is, CMVs (10 μg) prepared from each of the SD rats treated with 4PBA (filled circle) and a vehicle (open circle) was subjected to 6% SDS PAGE and then to Western blotting.

FIG. 9 shows the effects of a treatment with 4PBA or octanoic acid on MDCKII cells that had expressed BSEP. MDCKII cells that had expressed a wild-type BSEP were treated with 1 mM 4PBA or octanoic acid for 24 hours. Thereafter, a cell surface fraction was isolated by a biotinylation method, and the obtained sample was subjected to 6% SDS-PAGE and then to Western blotting.

FIG. 10 shows the effects of treatments with 4PBA, propionic acid, butyric acid, octanoic acid, and decanoic acid, on the transcellular transport of [³H] TC that got across the apical membrane of an MDCKII monolayer culture that had expressed a wild-type BSEP. MDCKII monolayer culture cells that had expressed a wild-type BSEP were treated in the presence or absence of 1 mM 4PBA, propionic acid, butyric acid, octanoic acid, and decanoic acid, for 24 hours. Thereafter, the clearance of the [³H] TC transport that got across the apical membrane was measured. Each bar indicates the mean value of 3 times of measurement values±a standard error. A significant difference from a control by a Student's test (*P<0.05; **P<0.01)

FIG. 11 shows the results obtained by examining the effects of octanoic acid on the decomposition rate of BSEP existing on the surfaces of MDCKII cells. FIG. 11A shows the results obtained by analyzing the decomposition rate of a wild-type BSEP existing on the surface of the cells. MDCKII cells that had expressed a wild-type BSEP were treated in the presence or absence of 1 mM octanoic acid. The cell surface was biotinylated, and the cells were cultured in the presence or absence of 1 mM octanoic acid at 37° C. for the period of time shown in the figure. The remaining biotinylated protein was isolated using streptavidin beads, and was then separated by 6% SDS-PAGE, followed by Western blotting. FIG. 11B shows the results obtained by quantifying the band densities of the wild-type BSEPs shown in FIG. 11A. The band density was quantified by Image Gauge software, and it was indicated with a percentage with respect to the strength at 0 (zero) hour. The filled circle (control) represents the value of each BSEP remaining on the surfaces of MDCK II cells that were not treated with carboxylic acid. The open circle and the filled square represent the values of BSEPs remaining on the surfaces of MDCKII cells treated with 4PBA and octanoic acid, respectively. Each bar indicates the mean value of 3 times of experiments±a standard error.

### BEST MODE FOR CARRYING OUT THE INVENTION

In Formula (1), R represents a hydrogen atom, a methyl group, or a phenyl group non-substituted or having a substituent; X represents a hydrogen atom or an alkali metal atom; and n represents an integer of 1 to 8. In this case, preferably, R is a methyl group, and n is an integer of 1, 2, 6, or 8.

Otherwise, in Formula (1), R is represented by the following formula (wherein R₁ represents a hydrogen atom or an alkyl group containing 1 to 5 carbon atoms); X represents a hydrogen atom or an alkali metal atom; and n represents an integer of 1 to 8.

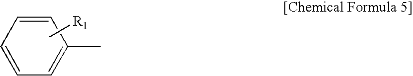

In this case, preferably, R₁ is a hydrogen atom, and n is 3.

The compound represented by Formula 1 of the present invention can be produced by a method known to persons skilled in the art. Alternatively, a commercially available product may also be purchased as such compound (for example, from Scandinavian Formulas Inc.).

The present invention provides an agent and a method for maintaining or promoting the stability of a membrane protein on a cell membrane.

The term “membrane protein” is used herein to mean a protein that constitutes a biological membrane. The membrane protein is preferably a transporter, and particularly preferably an ABC transporter. Examples of such transporter include MDR1, MRP1, MRP2, MRP3, MRP4, MRP5, MRP6, BCRP, OCT1, OCT2, OCTN2, OAT-K1, OATP-A, OATP-2, LST1, and BSEP. Such transporter is most preferably BSEP (bile salt export pump) (for example, see SEQ ID NO: 1 and SEQ ID NO: 2 as the sequences of human BSEPs). Moreover, as with such BSEP, MRP2, which is an ABC transporter expressed on the lateral membrane of the biliary canaliculus of a liver cell, is also preferable as a transporter used in the present invention. Such MRP2 is stabilized by phenylbutyric acid, and abnormality in the functions thereof provokes jaundice and causes liver function failure.

The agent according to the present invention adopts the form of a pharmaceutical composition that does not affect living bodies, and it can be used as an agent for treating abnormality in the functions of a membrane protein, and particularly, a transporter. In general, such composition comprises a pharmaceutically acceptable carrier, as well as the compound of the present invention.

Such “pharmaceutically acceptable carrier” comprises a solvent, a dispersion medium, a coating agent, an antibacterial agent, an antifungal agent, an agent for acting on isotonicity to retard adsorption, and an analogue thereof. Such carrier should be suitable for pharmaceutical administration. Preferred examples of the carrier and a component for diluting the carrier include, but are not limited to, water, a normal saline, a finger solution, a dextrose solution, collagen, human serum albumin, an organic solvent, collagen, polyvinyl alcohol, polyvinylpyrrolidone, a carboxy vinyl polymer, sodium alginate, carboxymethyl starch sodium, pectin, xanthan gum, gum Arabic, casein, gelatin, agar, glycerin, propylene glycol, polyethylene glycol, Vaseline, paraffin, stearyl alcohol, stearic acid, human serum albumin, mannitol, sorbitol, and lactose. In addition, water-insoluble media such as liposome and nonvolatile oil may also be used. Moreover, the composition may also comprise a specific compound for protecting or promoting the activity of compound of the present invention.

The pharmaceutical composition according to the present invention is formulated such that it can be administered via an intravenous, intradermal, subcutaneous, oral (including inhalation, for example), percutaneous, or transmucosal administration route, and such that it is compatible with a therapeutically appropriate administration route. A solution or a suspension applied to non-oral, intradermal, or subcutaneous administration may comprise a sterilized diluent such as water for injection, a normal saline, a nonvolatile oil, polyethylene glycol, glycerin, propylene glycol, other synthetic solvents, preservatives such as benzyl alcohol or other methylparabens, antioxidants such as ascorbic acid or sodium bisulfite, soothing agents such as benzalkonium chloride or procaine hydrochloride, chelating agents such as ethylenediaminetetraacetic acid (EDTA), buffers such as acetate, citrate, or phosphate, and agents for controlling osmotic pressure, such as sodium chloride or dextrose. However, components comprised in such solution or suspension are not limited thereto.

The pH may be controlled with an acid such as hydrochloric acid or sodium hydroxide, or a base. A non-oral specimen is placed in an ampule, a disposable syringe made of glass or plastic, or a vial used for multiple administration.

A pharmaceutical composition suitable for injection comprises a sterilized injectable solution or dispersion medium, a sterilized (water-soluble) aqueous solution or dispersion medium to be prepared when is used, and sterilized powders. As for intravenous administration, a suitable carrier comprises a normal saline, bacteriostatic water, or a phosphate buffered saline (PBS). When a pharmaceutical composition is used as an injection, it must be sterilized. In addition, for administration using a syringe, the pharmaceutical composition must have sufficient flowability. During formulation and preservation, the composition must be stable against chemical change, corrosion, and the like. Thus, it is necessary to prevent contamination from microorganisms such as bacteria and fungi. As a carrier, there may be used a solvent or a dispersion medium, which comprises water, ethanol, polyol (glycerol, propylene glycol, liquid polyethylene glycol, etc.), and an appropriate mixture. For example, using a coating agent such as lectin, a dispersion medium maintaining a necessary particle size, and a surfactant, moderate flowability can be maintained. Various antibacterial agents and antifungal agents, such as paraben, chlorobutanol, phenol, ascorbic acid, and thimerosal, can be used to prevent contamination caused by microorganisms. Moreover, the composition may also comprise agents maintaining isotonicity including sugar, polyalcohol such as mannitol or sorbitol, and sodium chloride. A composition, which is able to retard adsorption, comprises agents such as aluminum monostearate and gelatin.

A sterilized injectable solution is prepared by using alone a necessary ingredient or combining such necessary ingredient with other ingredients, then adding a necessary amount of active compound into a suitable solvent, and then sterilizing the obtained solution. In general, a dispersion medium is prepared by incorporating an active compound into a sterilized medium containing a basic dispersion medium and the aforementioned other necessary ingredients. A method of preparing sterilized powders used to prepare such sterilized injectable solution includes vacuum drying and freeze-drying, which are applied to prepare powders comprising any desired ingredients derived from an active ingredient and a sterilized solution.

An oral composition comprises an inactive diluent and a carrier that does not affect a living body even if it is incorporated into the body. Such oral composition is encapsulated into a capsule made of gelatin, or it is compressed into a tablet, for example. For oral therapy, an active compound is used together with an excipient, and it is used in the form of a tablet, a troche, or a capsule. In addition, such oral composition can also be prepared using a fluid carrier. The composition contained in the fluid carrier is orally applied. Moreover, the oral composition may also comprise a binder and/or an adjuvant substance, and the like, which are pharmaceutically suitable.

A tablet, a pill, a capsule, a troche, and an analogue thereof may comprise any of the following ingredients, or compounds having properties similar thereto: excipients such as microcrystalline cellulose; binders such as gum Arabic, tragacanth, or gelatin; swelling agents such as starch, lactose, alginic acid, PRIMOGEL, or corn starch; lubricants such as magnesium stearate or STRROTES; slip additives such as colloidal silicon dioxide; sweeteners such as sucrose or saccharin; and aroma chemicals such as peppermint, methylsalicylic acid, or orange flavor.

The compound of the present invention can be prepared as a sustained-release preparation, such as a delivery system encapsulated into an implant or a microcapsule, using a carrier capable of preventing the compound from being rapidly eliminated from a body. There can be used a biodegradable, biocompatible polymer, such as ethylene vinyl acetate, poly acid anhydride, polyglycolic acid, collagen, polyorthoester, or polylactic acid. Such material can be easily prepared by persons skilled in the art. In addition, a liposome suspension can also be used as a pharmaceutically acceptable carrier. The types of a useful liposome is not limited. Such liposome is prepared as a lipid composition containing phosphatidyl choline, cholesterol, and PEG-derived phosphatidyl-ethanol (PEG-PE), by passing through a filter with an appropriate pore size, such that it has a size appropriate to use. It is then purified by a reverse phase evaporation method.

In the prevention or treatment of a specific disease by the compound of the present invention, an appropriate dose level of the present compound depends on the condition of a patient to which the compound is to be administered, an administration method, and the like. Persons skilled in the art could easily optimize such dose level.

In the case of injection administration, the composition is preferably administered at a dose from approximately 0.1 μg/kg to approximately 500 mg/kg per body weight of a patient per day, for example. In general, the composition may be administered once or divided over several administrations. The dose level is preferably from approximately 0.1 μg/kg to approximately 250 mg/kg per day, and more preferably from approximately 0.5 μg/kg to approximately 100 mg/kg per day.

In the case of oral administration, the composition is preferably provided in the form of a tablet containing 1.0 to 1,000 mg of an active ingredient. Preferably, the amount of such active ingredient is 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, or 1,000.0 mg. The compound is administered at a frequency of 1 to 4 times a day, and preferably once or twice a day.

A pharmaceutical composition or a preparation must be constituted with a uniform unit dosage in order to secure a constant dosage. The “unit dosage” means a single unit, which contains a single dose effective for treating a patient and which is formulated together with a pharmaceutically acceptable carrier. The determination of the unit dosage of the invention is influenced, for example, by the physical and chemical characteristics of a compound to be formulated, the expected therapeutic effects thereof, and points of concern in formulation typical for the compound.

The pharmaceutical composition of the present invention may adopt the form of a kit, and it may be placed in a container or a package, together with an instruction manual for administration. In a case in which the pharmaceutical composition according to the present invention is provided in the form of a kit, different constituents of the pharmaceutical composition are packaged into different containers, and they are mixed immediately before use. Thus, several constituents are packaged in different containers because it makes possible to conserve them for a long period of time without losing the functions of such active constituents.

A reagent contained in a kit is supplied into several types of containers, in which constituents effectively maintain their activity for a long period of time and such constituents are neither adsorbed nor degenerated depending on the material of such container. For example, a sealed glass ampule contains a buffer that has been packaged under neutral unreactive gas such as nitrogen gas. Such ampule is constituted with a glass, an organic polymer such as polycarbonate or polystyrene, ceramic, metal, other suitable materials generally used to retain a reagent, or the like. Examples of such other suitable containers include: a simple bottle prepared from substances similar to those for ampule; and a packaging material whose internal portion has been lined with the foil of aluminum, alloy, etc. Other containers include a test tube, a vial, a flask, a bottle, a syringe, and a similar product thereof. Such container has a sterile access port, such as a bottle having a stopper that can be penetrated with a subcutaneous injection needle.

Moreover, an instruction manual is included with the kit. An instruction manual for a kit comprising the present pharmaceutical composition may be printed on a paper or other materials, and/or may be supplied as an electrically or electromagnetically readable medium, such as a floppy (registered trademark) disk, CD-ROM, DVD-ROM, a Zip disk, a videotape, and an audiotape. A detailed instruction manual may be actually attached into the kit. Otherwise, it may also be published on a webpage designated by a kit manufacturer or distributor, or be noticed via an E-mail.

Furthermore, the present invention also includes a method for preventing or treating a mammal that is affected with a disease associated with abnormality in the functions of a membrane protein, and particularly, a transporter, or that is likely to be affected with such disease.

The term “treatment” is used herein to mean the inhibition or alleviation of the progression of the pathologic condition of the disease of a mammal that is likely to be affected with or is affected with the aforementioned disease. This term is used herein to have a broad sense including not only therapeutic measures but also preventive measures.

The “mammal” used as a therapeutic target means any given animal belonging to mammals, and thus the type of such animal is not particularly limited. Examples of such mammal include: humans; pet animals such as a dog and a cat; and livestock animals such as a bovine, a swine, a sheep, and a horse. A particularly preferred “mammal” is a human.

### Examples

Hereafter, the present invention will be described in details in the following examples, in which the maintenance and promotion of the stability of BSEP as a transporter protein on a canalicular membrane will be analyzed using the phenylbutyrate as shown in Formula (3) below, propionate, butanoate, octanoate, and decanoate.

However, the following examples are not intended to limit the scope of the present invention.

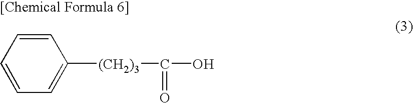

### Methods and Reagents

### 1. Reagents

Phenylbutyric acid (4PBA) and other carboxylic acids (propionic acid, butanoic acid, octanoic acid, and decanoic acid), which were at pharmaceutical grades, were purchased from Scandinavian Formulas Inc. [³H] taurocholic acid (2Ci/PA) was purchased from NEN Life Science Products. Antibodies against human BSEP, P-glycoprotein (P-gp) (C219), and dipeptidyl peptidase IV (DPPIV) were purchased from Santa Cruz Biotechnology, Signet, and BD Biosciences (CA), respectively. An antiserum against rBSEP was associated with an oligopeptide (the C-terminus of rBSEP; AYYKLVITGAPIS (SEQ ID NO: 3)), and it was produced using a rabbit. All other reagents used were at analytical grades. MDCKII cells were cultured in a DMEM medium (Invitrogen) containing 10% FBS, penicillin (100 U/ml) and streptavidin (100 U/ml), under conditions consisting of a temperature of 37° C., 5% CO₂, and a humidity of 95%.

### 2. Preparation of Recombinant Adenovirus

In order to prepare recombinant adenoviruses with human wild-type, E297G, and D482G BSEPs, the BD Adeno-X Adenoviral Expression system (BD Biosciences) was used (see Hayashi et al., Hepatology, 41: 916-924, 2005). The titer of such virus was measured using the Adeno-X Rapid Titer Kit (Clontech). As a control, a recombinant adenovirus having GFP was used.

3. Treatment with 4PBA and Octanoic Acid

MDCKII cells were dispersed on a 6-well plate, resulting in a density of 2.5×10⁵ cells/well. After completion of the culture for 24 hours, the confluent cells were infected with recombinant adenoviruses having the cDNAs of wild-type, E297G and D482G BSEPs, and GFP, at 200 MOI. A treatment with 4PBA or octanoic acid was carried out, while changing a treatment time and a treatment concentration. After completion of the treatment with 4PBA or octanoic acid, a crude membrane fraction was prepared (see Mita et al., Am J Physiol Gastrointest Liver Physiol 290: G550-G556, 2006). The obtained sample was separated by 6% SDS-PAGE, and Western blotting was then carried out (Hayashi et al., Hepatology, 41: 916-924, 2005). An antibody reaction was detected by ECL (Amersham Biosciences). The strength of a band indicating a mature BSEP was quantified by the Multi Gauge software Ver. 2.0 (Fujifilm).

### 4. Transcellular Transport Assay

MDCKII cells were dispersed on a Transwell membrane insert (pore size: 3 μM; Falcon) of a 24-well plate, resulting in a density of 1.5×10⁵ cells/insert. Two days after the culture, the confluent cells were infected with recombinant adenoviruses having the cDNAs of wild-type, E297G and D482G BSEPs, and GFP, at 50 MOI. After completion of the infection, the cells were cultured for 24 hours; and were then treated with 1 mM 4PBA or other carboxylic acids (propionic acid, butanoic acid, octanoic acid, and decanoic acid). After completion of the treatment for 24 hours, a transcellular transport assay was carried out (see Mita et al., Am J Physiol Gastrointest Liver Physiol 290: G550-G556, 2006).

The apparent efflux clearance across the apical membrane (PS_{apical}) was calculated by dividing a steady rate of the transcellular transport of [³H] TC for 2 hours by the intracellular concentration of the [³H] TC at the termination of the experiment (2 hours later).

### 5. Biotinylation of Cell Surface and Measurement of Decomposition Rate of Cell Surface-Expressed Protein

MDCKII cells were dispersed on a 6-well plate, resulting in a density of 2.5×10⁵ cells/well. After completion of the culture for 24 hours, the confluent cells were infected with recombinant adenoviruses having the cDNAs of wild-type, E297G and D482G BSEPs, and GFP, at 200 MOI. After completion of the infection, the cells were cultured for 24 hours. Thereafter, the cultured cells were treated with 1 mM 4PBA or octanoic acid. After completion of the treatment for 24 hours, the cells were biotinylated (see Hayashi et al., Hepatology, 41: 916-924, 2005).

When the decomposition rate of a protein existing on the cell surface, before solubilization, the biotinylated MDCKII cells were incubated at 37° C. for a certain period of time in the presence or absence of 1 mM 4PBA or octanoic acid. The remaining biotinylated protein was isolated, and it was then subjected to 6% SDS-PAGE, followed by Western blotting.

6. Measurement of BSEP mRNA Level

MDCKII cells were dispersed on a 6-well plate, resulting in a density of 2.5×10⁵ cells/well. After completion of the culture for 24 hours, the confluent cells were infected with recombinant adenoviruses having the cDNAs of wild-type, E297G and D482G BSEPs, and GFP, at 50 MOI. After completion of the infection, the cells were cultured for 24 hours. Thereafter, the cultured cells were treated with 1 mM 4PBA. After completion of the treatment for 24 hours, RNA was isolated using ISOGEN (Wako Pure Chemical Industries, Ltd.) in accordance with an instruction manual included therewith. A DNaseI treatment was carried out at 37° C. for 1 hour, and a reverse transcription reaction was then carried out (see Hayashi et al., Hepatology, 41: 916-924, 2005). The BSEP mRNA level was measured by real-time quantitative PCR according to an ordinary method. The quantitative PCR was carried out using the following primers.

| | |
|---|---|
| BSEP: | |
| 5′-dAGTGGGGGAGCTGAATACAA-3′ | (SEQ ID NO: 4) |
| 5′-dCCAATGGTGGCTGCTCCAAT-3′ | (SEQ ID NO: 5) |
| GAPDH: | |
| 5′-AACGACCCCTTCATTGAC-3′ | (SEQ ID NO: 6) |
| 5′-TCCACGACATACTCAGCAC-3′ | (SEQ ID NO: 7) |

It is to be noted that the expression of a BSEP gene in each reaction was standardized by the expression of GAPDH.

7. Treatment with Actinomycin D (ActD) and Cycloheximide (CHX)

MDCKII cells were dispersed on a 6-well plate, resulting in a density of 2.5×10⁵ cells/well. After completion of the culture for 24 hours, the confluent cells were infected with recombinant adenoviruses having the cDNAs of wild-type, E297G and D482G BSEPs, and GFP, at 200 MOI. After completion of the infection, the cells were cultured for 36 hours. Thereafter, in order to inhibit mRNA synthesis, the cells were treated in the presence or absence of 5 μg/ml actinomycin D (Sigma). Otherwise, in order to inhibit protein synthesis, the cells were treated in the presence or absence of 20 μg/ml cycloheximide (Sigma). After completion of the treatment for 2 hours, in the presence or absence of actinomycin D or in the presence or absence of cycloheximide, 4PBA was added to the cells to a concentration of 1 mM. At the time point of 6 hours (E297G and D482G BSEPs) or 8 hours (wild-type BSEP) after the 4PBA treatment, a crude membrane fraction was prepared. The obtained fraction was separated by 6% SDS-PAGE, and Western blotting was then carried out.

### 8. Brefeldin A Washing Experiment

MDCKII cells were dispersed on a 6-well plate, resulting in a density of 2.5×10⁵ cells/well. After completion of the culture for 24 hours, the confluent cells were infected with recombinant adenoviruses having the cDNAs of wild-type, E297G and D482G BSEPs, and GFP, at 200 MOI. After completion of the infection, the cells were cultured for 12 hours. Thereafter, in order to inhibit the transport of BSEP from the endoplasmic reticulum to the Golgi body, the cells were treated in the presence or absence of 5 μg/ml brefeldin A (Sigma). After completion of the treatment for 2 hours, in the presence or absence of brefeldin A, 4PBA was added to the cells to a concentration of 1 mM. At the time point of 12 hours after the 4PBA treatment, a crude membrane fraction was prepared. The obtained fraction was separated by 6% SDS-PAGE, and Western blotting was then carried out.

### 9. Animal Experiment

Male SD rats (6- to 7-week-old) were purchased from Nippon SLC. All the animals were bred under day-night reversal conditions. They were treated under standard conditions based on the protection of animals. Feed and water were given without any limitation.

It is to be noted that the present study was carried out in accordance with the guidelines determined by the animal experiment committee in the university. 4PBA or a vehicle was given to each SD rat via a tube, for a certain period of time (5, 10, and 15 days), at various doses (0.2, 0.6, and 2.4 kg/day). After completion of the 4PBA treatment, a [³H] TC injection experiment was carried out in vivo (see Hirano et al., Mol Pharmacol 68: 800-807. 2005). [³H] TC dissolved in a normal saline was injected into each rat, via a cannula inserted into the femoral vein thereof, at a rate of 70 ng/min/kg for 90 minutes. Blood and bile were collected for a certain period of time. Radioactivity in plasma, bile, and liver was measured.

### 10. Pharmacokinetic Analysis

The total clearance of plasma (CLₜₒₜₐₗ), the clearance of bile standardized with circulating plasma (CL_{bile, plasma}), and the clearance of bile standardized with the concentration in the liver (CL_{bile, liver}) were calculated using the following formulae:

CLₜₒₜₐₗ=I/C_{ss, plasma};

CL_{bile, plasma}=V_{ss, bile}/C_{ss, plasma}; and

CL_{bile, liver}=V_{ss, bile}/C_{ss, liver}

(wherein “I,” “C_{ss, plasma},” “V_{ss, bile}” and “C_{ss, and liver}” represent, respectively, an injection rate (ng/min/kg), a plasma concentration in a steady state (ng/min/kg), a bile elimination rate in a steady state (ng/min/kg), and a mean liver concentration (ng/ml)). The plasma concentration in a steady state (C_{ss, plasma}) indicates the mean value of plasma [³H] TC concentrations at time points of 15, 30, 60, and 90 minutes. The bile elimination rate (V_{ss, bile}) indicates the mean value of the bile elimination rates of [³H] TC at time points of 10-15 minutes, 25-30 minutes, 55-60 minutes, and 85-90 minutes. The mean liver concentration (C_{ss, liver}) indicates the mean value of liver [³H] TC concentrations at the termination of the in vivo experiment.

### 11. Transport Assay Using Canalicular Membrane Vesicles

0.6 kg/day 4PBA or a vehicle was given to a male SD rat (6- to 7-week-old) for 10 days, divided over 3 administrations. Canalicular membrane vesicles were prepared from the liver of the rat (Akita et al., Biochim Biophys Acta, 1511: 7-16, 2001), and they were then used in Western blotting and a transport assay. Such transport assay was carried out by a rapid filtration method (Hayashi et al., Biochim Biophys Acta, 1738: 54-62, 2005).

### Results

### 1. Upregulation of BSEP on Cell Surface Mediated by 4PBA or Octanoic Acid

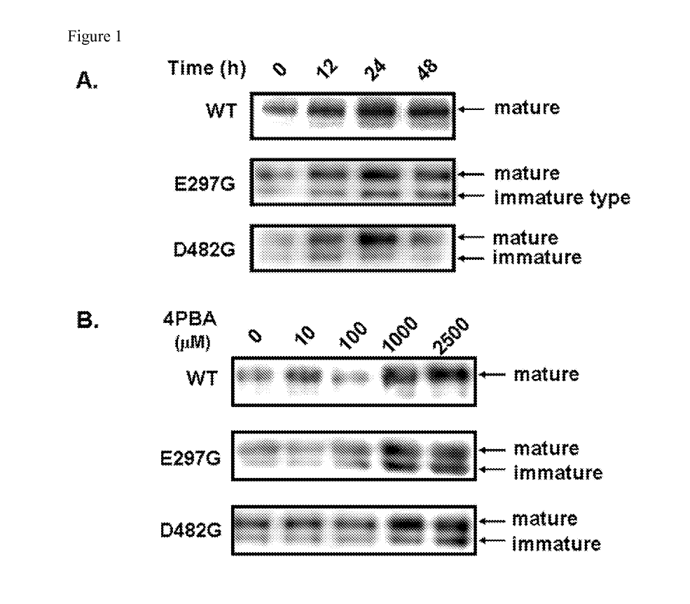

MDCKII cells that had expressed wild-type, E297G and D482G BSEPs were treated with 4PBA (FIGS. 1A and 1B). To date, the inventors have reported that the molecular weight of a mature BSEP existing on the membrane surface and the molecular weight of an immature BSEP existing in the endoplasmic reticulum were found to be 173 kDa and 150 kDa, respectively (Hayashi et al., Hepatology, 41: 916-924, 2005). Western blotting was performed on a crude membrane fraction to examine the expression level of such mature BSEP, so as to analyze the effects of 4PBA. With regard to the expression level of the mature BSEP, the expression level of wild-type BSEP as well as the expression levels of E297G and D482G BSEPs were changed by the 4PBA treatment. The optical treatment condition was a treatment with 1 mM 4PBA for 24 hours (clinically applicable amount). As a result of the treatment under such conditions, the expression levels of the wild-type, E297G and D482G BSEPs were increased by 2.5 to 3 times.

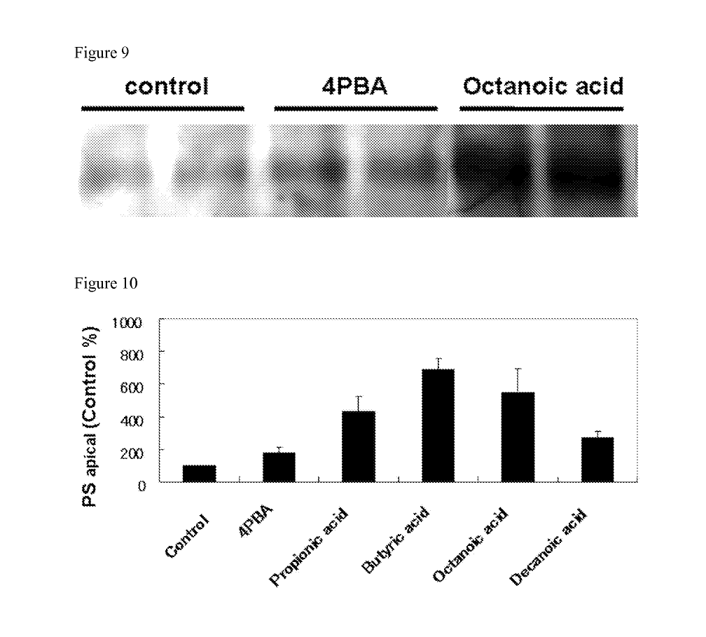

In addition, in a case in which the wild-type BSEP was treated with 1 mM octanoic acid for 24 hours as well, an increase in the expression level was observed (FIG. 9).

Subsequently, an increase in the expression of BSEP on the cell surface by 4PBA or other carboxylic acids was analyzed by a transcellular transport assay and a cell surface biotinylation method.

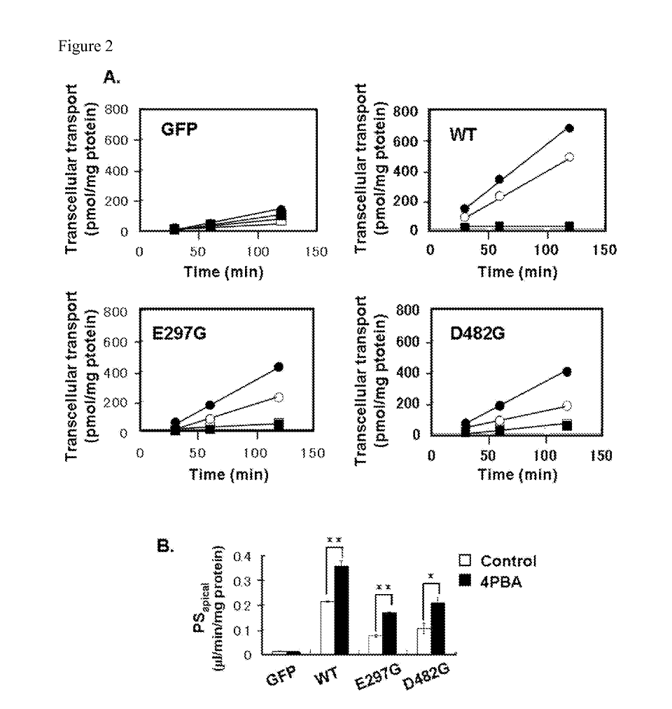

In the transcellular transport assay, the transcellular transport level of [³H] TC across a monolayer culture of MDCKII was measured in respect of the functions of BSEP mediating the efflux of the [³H] TC to the apical side (FIG. 2). Such [³H] TC transport directed to the apical side was observed in all of the cells that had expressed the wild-type, E297G and D482G BSEPs. In contrast, such [³H] TC transport was hardly observed in the cells that had expressed GFP.

The efflux of [³H] TC that got across the monolayer cells of MDCKII expressing the wild-type, E297G and D482G BSEPs, from the base side to the apical side, was increased by 1.5 times, 2.5 times and 3 times, respectively, as a result of the 4PBA treatment (1 mM, 24 hours). On the other hand, such effects were not observed in the cells expressing GFP. Moreover, 4PBA did not influence on the efflux of the [³H] TC from the apical side to the base side in all types of MDCKII cells.

Furthermore, in a case in which the wild-type BSEP was treated with other carboxylic acids (propionic acid, butanoic acid, octanoic acid, and decanoic acid), the efflux of the [³H] TC from the base side to the apical side was increased, as in the case of treating with 4PBA (FIG. 10).

In order to directly confirm the effects of the 4PBA treatment on the functions of BSEP, a kinetic parameter essential for the functions of BSEP, PS_{apical}, was calculated (Hayashi et al., Hepatology, 41: 916-924, 2005; Mita et al., Drug Metab Dispos, 34: 1575-1591, 2006) (FIG. 2B). Such PS_{apical} value was increased in MDCKII cells expressing the wild-type, E297G and D482G BSEPs as a result of the 4PBA treatment, but no changes were found in the cells expressing GFP. A BSEP-dependent PS_{apical} value (PS_{apical, BSEP}) in each of the MDCKII cells expressing the wild-type, E297G and D482G BSEPs was calculated by subtracting the PS_{apical} value of the MDCKII cells expressing GFP from the PS_{apical} value of each of the MDCKII cells expressing the wild-type, E297G and D482G BSEPs. The PS_{apical, BSEP} values of the MDCKII cells expressing the wild-type, E297G and D482G BSEPs were increased by 1.7 times, 3.0 times and 2.8 times, respectively, as a result of the 4PBA treatment under optical conditions (1 mM, 24 hours).

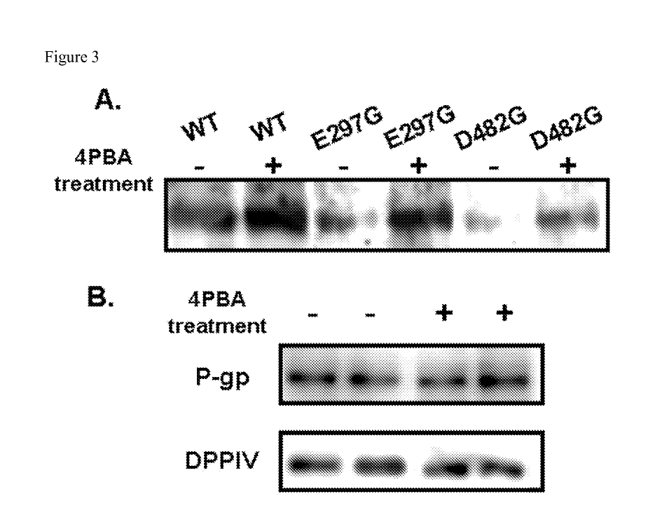

Moreover, the expression of wild-type and mutant BSEPs on the cell surface was analyzed by a cell surface biotinylation method using MDCKII cells (FIG. 3A). The expression levels of wild-type, E297G and D482G BSEPs on the cell surface were increased by 1.8 times, 3.1 times and 2.6 times, respectively, as a result of the 4PBA treatment under optical conditions (1 mM, 24 hours). On the other hand, the expression of extrinsic P-gp and the expression of intrinsic dipeptidyl peptidase IV (DPPIV) was not influenced by such 4PBA treatment (FIG. 3B). The increased expression level of BSEP on the cell surface by the 4PBA treatment was equivalent to the increased level of PS_{apical, BSEP}. This result suggested that the 4PBA treatment at a clinically applicable level increases the expression levels of the wild-type, E297G and D482G BSEPs on the cell surface, thereby promoting the transport capacity of BSER

2. Influence of 4PBA on Transcription and Translation of BSEP with Respect to Increase in Expression of BSEP on Cell Surface

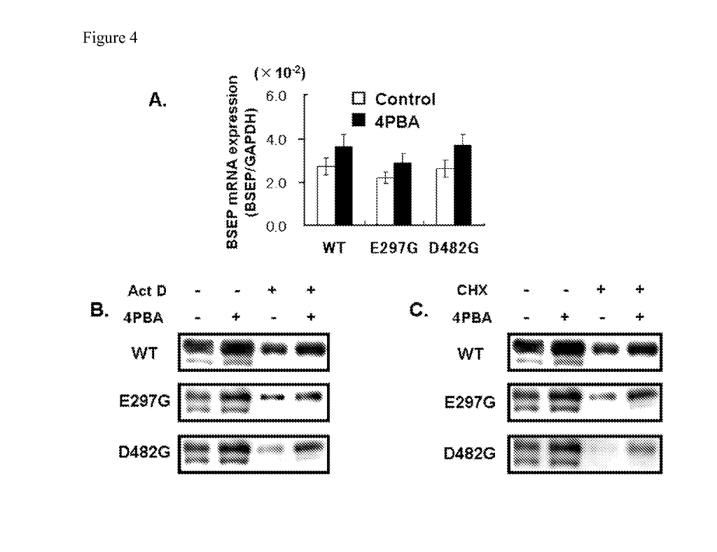

A mechanism whereby the 4PBA treatment increases the expression level of BSEP on the cell surface was analyzed. It was likely that 4PBA increases the transcription or translation level of a BSEP protein, so as to increase the expression level of the BSEP protein on the cell surface. The expression level of BSEP mRNA in MDCKII cells with or without the 4PBA treatment was measured by quantitative PCR. The expression level of such BSEP mRNA was standardized by the expression level of GAPDH mRNA that is not influenced by 4PBA. The expression levels of the mRNAs of wild-type, E297G and D482G BSEPs were slightly increased by the 4PBA treatment. However, such increased values were not statistically significant (P=0.10 (wild-type), 0.20 (E297G and D482G); FIG. 4A).

Thereafter, with regard to an increase in the expression of BSEP, there were used MDCKII cells that had been pre-treated with actinomycin D and cycloheximide, and the influence of the 4PBA treatment on the transcription and translation of the BSEP was analyzed (FIGS. 4B and 4C). Transcription inhibition by actinomycin D and translation inhibition by cycloheximide did not affect an increase in the expression of a mature BSEP due to 4PBA in the MDCKII cells that had expressed the wild-type, E297G and D482G BSEPs. These results demonstrated that a post-translation mechanism mainly contributes to an increase in the expression of BSEP on the cell surface due to 4PBA.

### 3. Influence of 4PBA Treatment on Maturation of BSEP

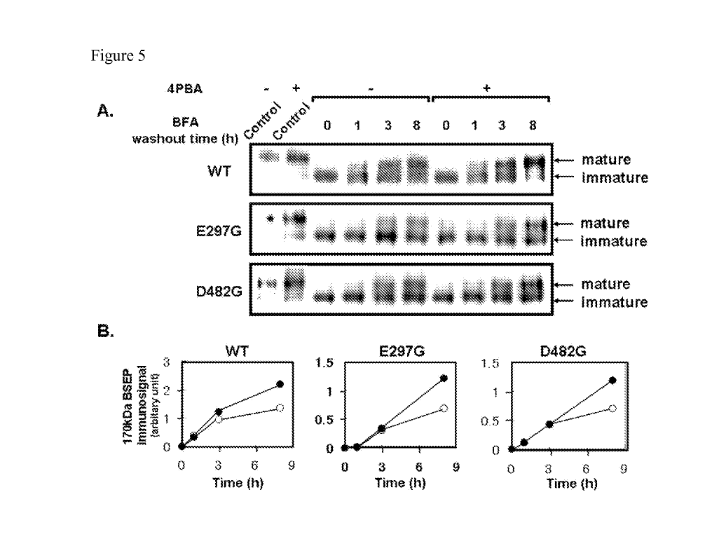

The promotion of a process of converting an immature BSEP existing in the endoplasmic reticulum to a mature BSEP existing on the cell surface is considered to be a post-translation mechanism that may be associated with an increase in the expression of the mature BSEP on the cell surface. In order to evaluate the effects of the 4PBA treatment on the maturation of a BSEP, the transport of the BSEP from the endoplasmic reticulum to the Golgi body was inhibited with brefeldin A (BFA), the BFA was then washed out, and the maturation rate of the BSEP on MDCKII cells was measured. BFA is an agent for inducing the accumulation of an immature BSEP existing in the endoplasmic reticulum (FIGS. 5A and 5B). The maturation rate was evaluated by washing out such BFA and then measuring an increased amount of the band density of a mature BSEP over time according to Western blotting. The band density of the mature BSEP was calculated by subtracting the band density at 0 (zero) hour from the band density at each measurement time (FIG. 5B). The expression levels of wild-type, E297G and D482G, immature BSEPs existing in the endoplasmic reticulum, immediately after the washing out of BFA, were almost the same, regardless of the presence or absence of the 4PBA treatment (FIG. 5A; hour 0). In addition, in the case of mature BSEPs, the expression levels were not changed until 3 hours after the washing out of BFA, regardless of the presence or absence of 4PBA. Three hours after the washing out of BFA and thereafter, however, such expression levels were linearly increased. At the time point 8 hours after the washing out of BFA, the MDCKII cells treated with 4PBA were compared with the MDCKII cells that had not been treated with 4PBA. As a result, the expression level of a mature BSEP in the MDCKII cells treated with 4PBA was higher than the same expression level in the MDCKII cells that had not been treated with 4PBA. This result suggested that the 4PBA treatment stabilizes such mature wild-type, G297G and D482G BSEPs without promoting the maturation of such BSEPs.

### 4. Extension of Half-Life of BSEP Existing on Cell Surface by 4PBA or Octanoic Acid

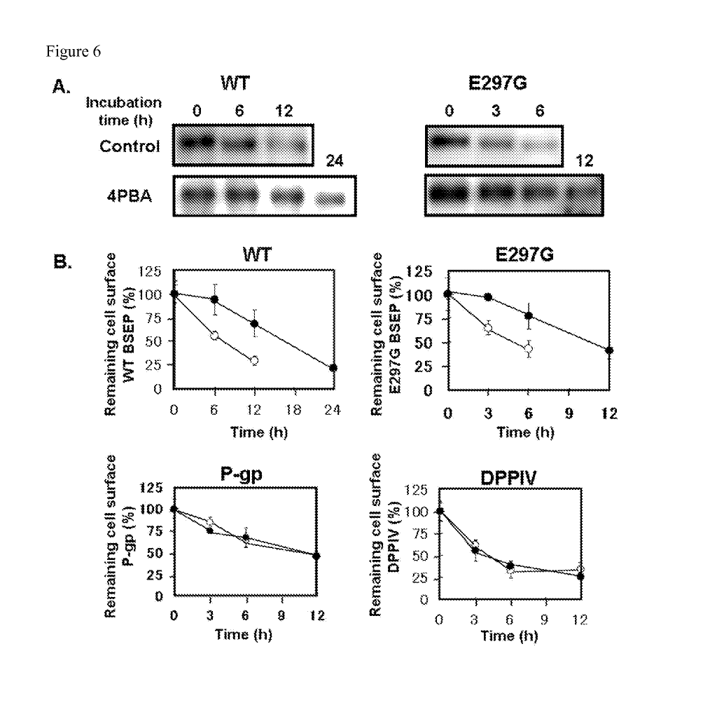

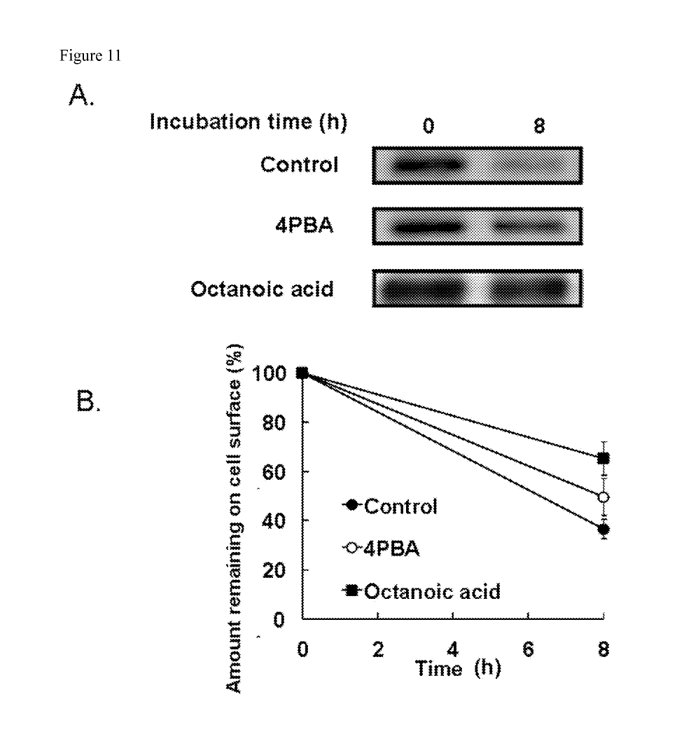

BSEP existing on the cell surface is circulated between a canalicular membrane and an intracellular compartment, and it is finally eliminated through a proteolytic pathway. Accordingly, it was likely that an increase in the expression of the BSEP on the cell surface is induced by blocking its entry to such proteolytic pathway. Thus, in order to analyze whether or not the entry of BSEP existing on the cell surface to such proteolytic pathway is inhibited by the 4PBA treatment, the decomposition rates of wild-type, E297G and D482G BSEPs on the surfaces of MDCKII cells expressing such BSEPs were measured according to a biotin-labeling method. The half-lives of the wild-type and E297G BSEPs existing on the cell surface were prolonged by 1.8 times and 2.5 times, respectively, by the 4PBA treatment (FIGS. 6A and 6B). Moreover, in the case of the wild-type BSEP, it was also treated with octanoic acid instead of 4PBA. In this case also, the same results were obtained (FIG. 11).

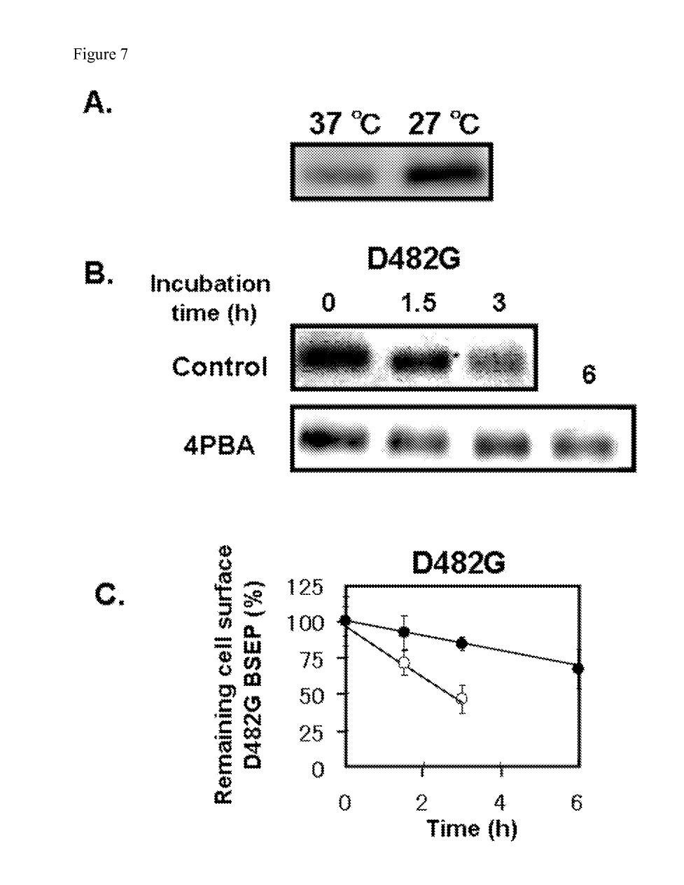

On the other hand, extrinsic P-gp and intrinsic dipeptidyl peptidase IV (DPPIV) were not influenced by such 4PBA treatment (FIGS. 6A and 6B). Since the expression level of a D482G BSEP was low, the decomposition rate of the D482G BSEP existing on the cell surface could not be detected under the same measurement conditions as those for the wild type and E297G BSEPs Hence, with regard to such D482G BSEP, the effects of a low-temperature treatment, known to correctly adjust the transport a mutant misfolded protein, was analyzed (Denning et al., Nature 358: 761-764, 1992; Plass et al., J Hepatol 40: 24-30, 2004) (FIG. 7A). When the cells were cultured under low-temperature conditions (27° C.) for 24 hours, the expression of the D482G BSEP on the cell surface was increased by 4 times. Thereafter, the decomposition rate of the D482G BSEP existing on the cell surface was measured at 37° C. As a result, the half-life thereof on the cell surface was increased by 3.3 times as a result of the 4PBA treatment (FIGS. 7B and 7C). From this result, it became clear that the maturation of BSEP is stabilized by the treatment with 4PBA (FIGS. 5A and 5B).

### 5. Upregulation of BSEP Expressing on Canalicular Membrane Under In-Vivo Conditions by 4PBA

The functional expression of wild-type, E297G and D482G BSEPs on the surfaces of MDCKII cells can be increased by a treatment with 4PBA (FIGS. 2 and 3). Thus, in order to analyze whether 4PBA has the same effects even in vivo, an experiment was carried out using SD rats. The dose applied to such SD rats was determined according to the dose approved to be used to children (0.45-0.6 g/kg/day). The rats were repeatedly treated with 4PBA at a dose of 0.6 k/kg/day for 10 days, and a [³H] TC injection experiment was then carried out. In terms of the C_{ss, plasma} and V_{ss, bile} of [³H] TC, there was found no significant difference between vehicle-treated SD rats and 4PBA-treated SD rats. However, the C_{ss, liver} of [³H] TC in the 4PBA-treated SD rats was 1/2 of the C_{ss, liver} of [³H] TC in the vehicle-treated SD rats (Tables 1 and 2). Accordingly, in terms of CLₜₒₜₐₗ and CL_{bile, plasma} with respect to [³H] TC, there was found no significant difference between the vehicle-treated SD rats and the 4PBA-treated SD rats. On the contrary, the CL_{bile, liver} with respect to the [³H] TC of the 4PBA-treated rats was 2 times greater than that of the vehicle-treated rats. These results suggested that the elimination of bile of [³H] TC via a canalicular membrane is promoted.

In order to analyze optical conditions for treating 4PBA, a [³H] TC injection experiment was carried out using various times and doses (Tables 1 and 2). As a result, the optical conditions were found to be a treatment with 4PBA at a dose of 0.6 g/kg/day for 10 to 15 days. No clear side effects were recognized under all conditions.

TABLE 1

| | | 4PBA | 4PBA | 4PBA |
|---|---|---|---|---|
| | Vehicle | (5 days) | (10 days) | (15 days) |
| | (N = 9) | (N = 6) | (N = 9) | (N = 3) |
| C_{ss, plasma} | 2.0 ± 0.2 | 2.3 ± 0.3 | 2.0 ± 0.2 | 2.1 ± 0.2 |
| (ng/ml) | | | | |
| C_{ss, liver} | 5.1 ± 0.6 | 3.1 ± 0.5^{xx} | 2.6 ± 0.5^{xxx} | 2.3 ± 0.2^{xx} |
| (ng/ml) | | | | |
| V_{ss, bile} | 64.4 ± 2.1 | 64.9 ± 7.8 | 67.9 ± 6.8 | 68.9 ± 2.4 |
| (ng/min/kg) | | | | |
| CLₜₒₜₐₗ | 29.4 ± 3.8 | 26.6 ± 3.8 | 30.7 ± 3.7 | 27.5 ± 2.7 |
| (ml/min/kg) | | | | |
| CL_{bile, plasma} | 31.4 ± 4.2 | 32.3 ± 8.5 | 32.3 ± 2.9 | 33.2 ± 2.0 |
| (ml/min/kg) | | | | |
| CL_{bile, liver} | 14.2 ± 3.0 | 21.7 ± 3.0^{x} | 29.3 ± 6.1^{xxx} | 29.9 ± 1.5^{xxx} |
| (ml/min/kg) | | | | |

Data are indicated by a mean value ± a standard error.
A significant difference from vehicle-treated SD rats by Student's t-test (*, P < 0.05; **, P < 0.01; ***, P < 0.001)

TABLE 2

| | | 4PBA (0.2 | 4PBA (0.6 | 4PBA (2.4 |
|---|---|---|---|---|
| | Vehicle | g/kg/day) | g/kg/day) | g/kg/day) |
| | (N = 9) | (N = 3) | (N = 9) | (N = 3) |
| C_{ss, plasma} | 2.0 ± 0.2 | 2.6 ± 0.2 | 2.0 ± 0.2 | 3.3 ± 0.7 |
| (ng/ml) | | | | |
| C_{ss, liver} | 5.1 ± 0.6 | 3.8 ± 0.7 | 2.6 ± 0.5^{xxx} | 5.9 ± 1.8 |
| (ng/ml) | | | | |
| V_{ss, bile} | 64.4 ± 2.1 | 66.5 ± 1.6 | 67.9 ± 6.8 | 73.3 ± 6.4 |
| (ng/min/kg) | | | | |
| CLₜₒₜₐₗ | 29.4 ± 3.8 | 26.6 ± 3.8 | 30.7 ± 3.7 | 29.6 ± 3.5 |
| (ml/min/kg) | | | | |
| CL_{bile, plasma} | 31.4 ± 4.2 | 26.3 ± 2.0 | 32.3 ± 2.9 | 23.9 ± 4.7 |
| (ml/min/kg) | | | | |
| CL_{bile, liver} | 14.2 ± 3.0 | 19.0 ± 3.9 | 29.3 ± 6.1^{xxx} | 19.2 ± 3.2 |
| (ml/min/kg) | | | | |

Data are indicated by a mean value ± a standard error.
A significant difference from vehicle-treated SD rats by Student's t-test (***, P < 0.001)

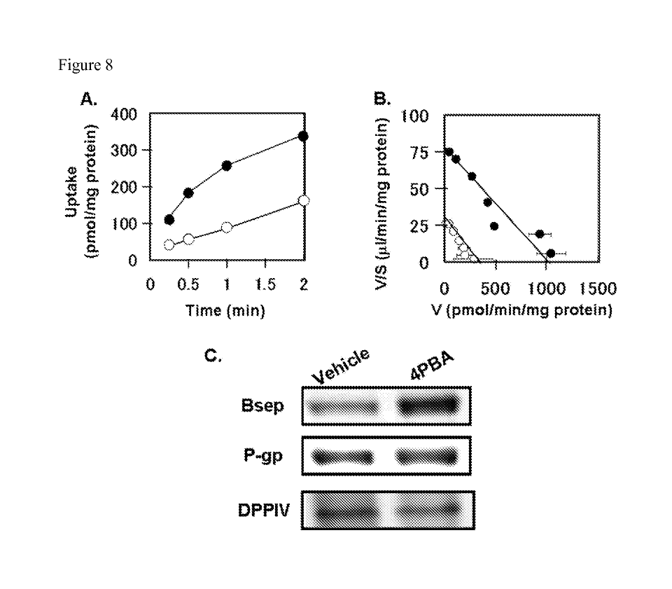

In order to analyze whether an increase in the expression of BSEP on a canalicular membrane is associated with the promotion of elimination of bile of [³H] TC via the canalicular membrane, using canalicular membrane vesicles (CMVs), a vesicle uptake experiment and Western blotting were carried out. The CMVs were prepared from SD rats that had been repeatedly treated with 4PBA or a vehicle at a dose of 0.6 g/kg/day for 10 days. The ATP-dependent uptake of [³H] TC by CMVs derived from the 4PBA-treated rats was linearly increased for 0.5 minutes. When compared with the same above uptake by CMVs derived from the vehicle-treated rats, the uptake level by the CMVs derived from the 4PBA-treated rats was 3 times higher (FIG. 8A). The initial rates of the uptake of [³H] TC by the CMVs derived from the 4PBA-treated rats and the CMVs derived from the vehicle-treated rats were indicated by single saturated ingredients having Kₘ values of 13.5±2.4 and 9.5±1.0 μM, respectively (FIG. 8B). The Vₘₐₓ value of [³H] TC (270±20) was increased to 1,000±110 by the 4PBA treatment (FIG. 8B). Also the following became clear. That is, Western blotting was performed on CMVs, and as a result, it was found that the expression level of BSEP was increased by 3.2 times as a result of the 4PBA treatment. In contrast, the expression levels of P-gp and DPPIV were not changed (FIG. 8C). These results suggest that the expression level of a functional BSEP on a canalicular membrane in an SD rat is increased by treating it with 4PBA in an amount clinically applicable to humans, and that the transport of bile acid via the canalicular membrane is thereby promoted.

### INDUSTRIAL APPLICABILITY

The stabilizing agent and stabilizing method of the present invention maintain and promote a membrane protein, and in particular, a transporter (for example, an ABC transporter) on a cell membrane. Thus, such stabilizing agent and stabilizing method are able to realize the promotion of the activity of such transporter (for example, an ABC transporter). Accordingly, the stabilizing agent and stabilizing method of the present invention greatly contribute to the development of an agent and a method for treating disease associated with abnormality in the functions of the transporter.

1. An agent for stabilizing a membrane protein, wherein it comprises, as an active ingredient, a compound represented by the following Formula (1), a pharmaceutically acceptable salt thereof, or a hydrate thereof: 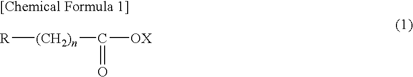
(wherein R represents a hydrogen atom, a methyl group, or a phenyl group non-substituted or having a substituent; X represents a hydrogen atom or an alkali metal atom; and n represents an integer of 1 to 8).

2. The stabilizing agent according to claim 1, wherein said R is a methyl group.

3. The stabilizing agent according to claim 2, wherein n is 1, 2, 6, or 8.

4. The stabilizing agent according to claim 1, wherein said R is a substituent represented by the following formula: 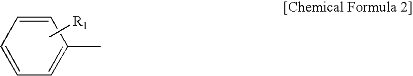
(wherein R₁ represents a hydrogen atom or an alkyl group containing 1 to 5 carbon atoms).

5. The stabilizing agent according to claim 4, wherein said R₁ is a hydrogen atom, and n is 3.

6. The stabilizing agent according to claim 1, wherein said membrane protein is a transporter.

7. The stabilizing agent according to claim 6, wherein said transporter is an ABC transporter.

8. The stabilizing agent according to claim 7, wherein said ABC transporter is BSEP.

9. The stabilizing agent according to claim 7, wherein said ABC transporter is MRP2.

10. A cholagogue, which comprises the stabilizing agent according to claim 8.


SEQUENCE LISTING (7 sequences)

SEQ ID NO: 1 - DNA, 3963 bp, Homo sapiens
atgtctgactcagtaattcttcgaagtataaagaaatttggagaggagaatgatggttttgagtcagataaatcatataataatgataagaaatcaaggttacaagatgagaagaaaggtgatggcgttagagttggcttctttcaattgtttcggttttcttcatcaactgacatttggctgatgtttgtgggaagtttgtgtgcatttctccatggaatagcccagccaggcgtgctactcatttttggcacaatgacagatgtttttattgactacgacgttgagttacaagaactccagattccaggaaaagcatgtgtgaataacaccattgtatggactaacagttccctcaaccagaacatgacaaatggaacacgttgtgggttgctgaacatcgagagcgaaatgatcaaatttgccagttactatgctggaattgctgtcgcagtacttatcacaggatatattcaaatatgcttttgggtcattgccgcagctcgtcagatacagaaaatgagaaaattttactttaggagaataatgagaatggaaatagggtggtttgactgcaattcagtgggggagctgaatacaagattctctgatgatattaataaaatcaatgatgccatagctgaccaaatggcccttttcattcagcgcatgacctcgaccatctgtggtttcctgttgggatttttcaggggttggaaactgaccttggttattatttctgtcagccctctcattgggattggagcagccaccattggtctgagtgtgtccaagtttacggactatgagctgaaggcctatgccaaagcaggggtggtggctgatgaagtcatttcatcaatgagaacagtggctgcttttggtggtgagaaaagagaggttgaaaggtatgagaaaaatcttgtgttcgcccagcgttggggaattagaaaaggaatagtgatgggattctttactggattcgtgtggtgtctcatctttttgtgttatgcagtggccttctggtacggctccacacttgtcctggatgaaggagaatatacaccaggaacccttgtccagattttcctcagtgtcatagtaggagctttaaatcttggcaatgcctctccttgtttggaagcctttgcaactggacgtgcagcagccaccagcatttttgagacaatagacaggaaacccatcattgactgcatgtcagaagatggttacaagttggatcgaatcaagggtgaaattgaattccataatgtgaccttccattatccttccagaccagaggtgaagattctaaatgacctcaacatggtcattaaaccaggggaaatgacagctctggtaggacccagtggagctggaaaaagtacagcactgcaactcattcagcgattctatgacccctgtgaaggaatggtgaccgtggatggccatgacattcgctctcttaacattcagtggcttagagatcagattgggatagtggagcaagagccagttctgttctctaccaccattgcagaaaatattcgctatggcagagaagatgcaacaatggaagacatagtccaagctgccaaggaggccaatgcctacaacttcatcatggacctgccacagcaatttgacacccttgttggagaaggaggaggccagatgagtggtggccagaaacaaagggtagctatcgccagagccctcatccgaaatcccaagattctgcttttggacatggccacctcagctctggacaatgagagtgaagccatggtgcaagaagtgctgagtaagattcagcatgggcacacaatcatttcagttgctcatcgcttgtctacggtcagagctgcagataccatcattggttttgaacatggcactgcagtggaaagagggacccatgaagaattactggaaaggaaaggtgtttacttcactctagtgactttgcaaagccagggaaatcaagctcttaatgaagaggacataaaggatgcaactgaagatgacatgcttgcgaggacctttagcagagggagctaccaggatagtttaagggcttccatccggcaacgctccaagtctcagctttcttacctggtgcacgaacctccattagctgttgtagatcataagtctacctatgaagaagatagaaaggacaaggacattcctgtgcaggaagaagttgaacctgccccagttaggaggattctgaaattcagtgctccagaatggccctacatgctggtagggtctgtgggtgcagctgtgaacgggacagtcacacccttgtatgcctttttattcagccagattcttgggactttttcaattcctgataaagaggaacaaaggtcacagatcaatggtgtgtgcctactttttgtagcaatgggctgtgtatctcttttcacccaatttctacagggatatgcctttgctaaatctggggagctcctaacaaaaaggctacgtaaatttggtttcagggcaatgctggggcaagatattgcctggtttgatgacctcagaaatagccctggagcattgacaacaagacttgctacagatgcttcccaagttcaaggggctgccggctctcagatcgggatgatagtcaattccttcactaacgtcactgtggccatgatcattgccttctcctttagctggaagctgagcctggtcatcttgtgcttcttccccttcttggctttatcaggagccacacagaccaggatgttgacaggatttgcctctcgagataagcaggccctggagatggtgggacagattacaaatgaagccctcagtaacatccgcactgttgctggaattggaaaggagaggcggttcattgaagcacttgagactgagctggagaagcccttcaagacagccattcagaaagccaatatttacggattctgctttgcctttgcccagtgcatcatgtttattgcgaattctgcttcctacagatatggaggttacttaatctccaatgaggggctccatttcagctatgtgttcagggtgatctctgcagttgtactgagtgcaacagctcttggaagagccttctcttacaccccaagttatgcaaaagctaaaatatcagctgcacgcttttttcaactgctggaccgacaacccccaatcagtgtatacaatactgcaggtgaaaaatgggacaacttccaggggaagattgattttgttgattgtaaatttacatatccttctcgacctgactcgcaagttctgaatggtctctcagtgtcgattagtccagggcagacactggcgtttgttgggagcagtggatgtggcaaaagcactagcattcagctgttggaacgtttctatgatcctgatcaagggaaggtgatgatagatggtcatgacagcaaaaaagtaaatgtccagttcctccgctcaaacattggaattgtttcccaggaaccagtgttgtttgcctgtagcataatggacaatatcaagtatggagacaacaccaaagaaattcccatggaaagagtcatagcagctgcaaaacaggctcagctgcatgattttgtcatgtcactcccagagaaatatgaaactaacgttgggtcccaggggtctcaactctctagaggggagaaacaacgcattgctattgctcgggccattgtacgagatcctaaaatcttgctactagatgaagccacttctgccttagacacagaaagtgaaaagacggtgcaggttgctctagacaaagccagagagggtcggacctgcattgtcattgcccatcgcttgtccaccatccagaacgcggatatcattgctgtcatggcacagggggtggtgattgaaaaggggacccatgaagaactgatggcccaaaaaggagcctactacaaactagtcaccactggatcccccatcagt

SEQ ID NO: 2 - protein, 1321 aa, Homo sapiens [sequence omitted]

SEQ ID NO: 3 - protein, 13 aa, Homo sapiens
AYYKLVITGAPIS

SEQ ID NO: 4 - DNA, 20 bp, Artificial
  designed nucleotides
agtgggggagctgaatacaa

SEQ ID NO: 5 - DNA, 20 bp, Artificial
  designed nucleotides
ccaatggtggctgctccaat

SEQ ID NO: 6 - DNA, 18 bp, Artificial
  designed nucleotides
aacgaccccttcattgac

SEQ ID NO: 7 - DNA, 19 bp, Artificial
  designed nucleotides
tccacgacatactcagcac